(19) United States
(12) Patent Application Publication
(10) Pub. No.: US 2004/0259080 A1
(43) Pub. Date: Dec. 23, 2004

(54) Apparatus and method for increasing insect resistance in transgenic plants

(76) Inventors: Gary W. Felton, Port Malilda, PA (US); Yinong Yang, (US); Qin Wang, (US)

Correspondence Address: HEAD, JOHNSON & KACHIGIAN, 228 W 17TH PLACE, TULSA, OK, 74119, US

(21) Appl. No.: 09/956,326

(22) Filed: Sep. 18, 2001

Related U.S. Application Data
(63) Continuation-in-part of application No. 09/205,630, filed on Dec. 3, 1998, now Pat. No. 6,303,326.
(60) Provisional application No. 60/067,457, filed on Dec. 4, 1997.

(51) Int. Cl. C12Q 1/68; C07H 21/04; C12N 9/02

(52) U.S. Cl. 435/6; 435/69.1; 435/189; 435/320.1; 435/325; 536/23.2

(57) ABSTRACT

The present invention discloses a polynucleotide sequence for an insect salivary glucose oxidase enzyme and the amino acid sequence of the enzyme itself. It also provides recombinant polynucleotide vector systems designed to express the enzyme in a variety of host organisms. The invention also discloses a method for creating transgenic plants having increased resistance to insect predation resulting from the expression of the foreign glucose oxidase protein. The presence of the insect glucose oxidase enzyme triggers a plant's defensive mechanisms and results in increased resistance to insects.

### CROSS REFERENCE TO RELATED APPLICATIONS

This application is a continuation-in-part of U.S. patent application Ser. No. 09/205,630, filed Dec. 3, 1998, the teachings of which are expressly incorporated herein by reference, which is a continuation-in-part of U.S. provisional patent application Ser. No. 60/067,457, filed Dec. 4, 1997, the teachings of which are expressly incorporated herein by reference.

### BACKGROUND OF THE INVENTION

1. Field of the Invention

The present invention relates to a glucose oxidase enzyme isolated from the corn earworm Helicoverpa zea and the polynucleotide sequence that codes for it. More specifically, the invention. relates to recombinant polynucleotide vectors having a polynucleotide encoding a glucose oxidase enzyme isolated from an insect that may be inserted into a host cell or organism. The invention also relates to transgenic organisms having the polynucleotide sequence and expressing the polypeptide. Such vectors may be used to create transgenic plants that show increased resistance to a broad range of pathogens. The polynucleotide encoding the glucose oxidase may be inserted into a variety of commercial crop plants by means of a variety of recombinant vectors. The invention also relates to a recombinant antibody that may be used to detect the presence of the glucose oxidase enzyme

2. Prior Art

The techniques of genetic engineering have been successfully applied to the pharmaceutical industry, resulting in a number of novel products. It has become apparent that the same technologies can be applied on a large scale to the production of enzymes of value to other industries. The benefits of achieving commercially useful processes through genetic engineering are expected to include cost savings in enzyme production, productions of enzymes in organisms generally recognized as safe which are suitable for food products, and specific genetic modifications at the genomic level to improve enzyme properties, such as thermal stability and performance characteristics, as well as those which would increase the ease with which the enzyme can be purified.

Genetic engineering and biotechnology are becoming increasingly important in the food industry. New technology may be used to make staple crops such as wheat, corn, potatoes and tomatoes easier and cheaper to grow. Transgenic plants, plants having genes from other organisms inserted into their cells, may be created that grow faster, grow in non-ideal environments or are more resistant to plant pathogens and insects. Transgenic plants that are sturdier or have strengthened defense mechanisms offer significant advantages to farmers around the world. In order to create transgenic plants, however, more must be understood about a plant's defenses against insects and other pathogens.

As will be appreciated by those skilled in the art, very little is understood about many of the complex interactions of insects and plants. For example, defoliation by soybean loopers triggers systemic acquired resistance to stem canker disease and redcrown rot. Conversly, stem-girdling by three-cornered alfalfa hoppers predisposes the same plants to the same diseases. Thus, the role of insects in triggering resistance or susceptibility to both insects and phytopathogens is still under investigation.

It has recently been recognized that the oral secretions from some herbivores trigger plants to release chemicals that attract the natural enemies of the herbivores feeding on the plants. For example, β-glucosidase in the regurgitant of Pieris brassicae caterpillars elicits the release of volatile compounds from cabbage leaves. More recently a glutamine-linolenic acid conjugate named volicitin was isolated from the regurgitant of beet armyworms Spodoptera exigua and found to induce the release of volatile chemicals from corn seedlings.

Monsanto has reported that natural plant defense responses to pathogen infection involve the production of active oxygen species including hydrogen peroxide (H₂O₂). Monsanto obtained transgenic potato plants expressing a fungal gene encoding glucose oxidase, which generates H₂O₂ when glucose is oxidized. H₂O₂ levels were elevated in both leaf and tuber tissues of these transgenic plants. Monsanto provided evidence that the trangenically elevated H₂O₂ levels enhanced disease resistance in potatoes against a broad range of plant pathogens. They report that the elevated levels of H₂O₂ in transgenic plants having a foreign glucose oxidase enzyme significantly increases the concentration of salicylic acid in leaf tissue, although no significant change was detected in the levels of free salicylic acid. The mRNAs of two defense-related genes encoding the anionic peroxidase and acidic chitinase were also induced. The results suggest that constitutively elevated sublethal levels of H₂O₂ are sufficient to activate an array of systemic host defense mechanisms, and these defense mechanisms may be a contributing factor to the H₂O₂-mediated disease resistance in transgenic plants. Systemic defense mechanisms, as opposed to local defense mechanisms, enhance resistance throughout the entire plant. Systemic defense mechanisms are therefore very desirable.

The transgenic potato tubers exhibited strong resistance to a bacterial soft rot disease caused by Erwinia carotovora and disease resistance was sustained under both aerobic and anaerobic conditions of bacterial infection. This resistance to soft rot was apparently mediated by elevated levels of H₂O₂ because the resistance could be counteracted by an exogenously added H₂O₂ degrading catalase.

The transgenic plants with increased levels of H₂O₂ also exhibited enhanced resistance to potato blight caused by Phytophthora infestans. The development of lesions resulting from infection by P. infestans was significantly delayed in leaves of these plants. Thus, the expression of active oxygen species-generating enzyme in transgenic plants represents a novel approach for engineering broad-spectrum, systemic disease resistance in plants.

The Salk Institute in La Jolla, Calif. has also used a similar approach to produce disease resistant rice. The Australian Science Foundation CSIRO scientists have developed a disease resistant cotton using the same gene. U.S. Pat. No.5,094,951 discusses the production of a fungal glucose oxidase in recombinant bacterial systems.

While glucose oxidase may be used with various agricultural applications, it may also be used for other applications. For example, glucose oxidase has been used with various food applications. U.S. Pat. No. 5,085,873 shows a process for the treatment of a non-food product for assuring its microbial decontamination. U.S. Pat. No. 4,996,062 shows a glucose oxidase food treatment and storage method. U.S. Pat. No. 4,990,343 shows an enzyme product and method of improving the properties of dough and the quality of bread. U.S. Pat. No. 4,957,749 shows a process for removing oxygen in foodstuffs and in drinks. U.S. Pat. No. 4,929,451 shows a process for eliminating disagreeable odor from soya milk. U.S. Pat. No. 4,557,927 shows various food products and processes for producing the same. U.S. Pat. No. 3,804,715 shows a process for preparing sugar containing maltose of high purity. U.S. Pat. No. 3,767,531 shows a preparation of insolubilized enzymes and U.S. Pat. No. 4,675,191 shows a method for production of a low alcoholic wine.

Glucose oxidase may also be used for other applications including biomedical and biochemical. For example, glucose oxidase may be used in the glucose monitoring of blood, urine, etc. as discussed by J. A. Lott and K. Turner in “Evaluation of Trinder's glucose oxidase method for measuring glucose in serum and urine,” Clin. Chem. 21 (12): 1745-1760 (1975). Another example is found in the product TES-TAPE® Lilly Glucose Enzymatic Test Strips. Yet another example is shown in U.S. Pat. No. 5,304,468, which shows a reagent test strip and apparatus for determination of blood glucose.

Glucose oxidase may also have other medical uses, such as the development of anticancer and/or antitumor agents as reported by C. F. Nathan and Z. A. Cohn in “Antitumor Effects of Hydrogen Peroxide in Vivo,” J. Exp. Med, Vol. 154, 1539-1553 (1981) and by Samoszuk M. D. Ehrlich and E. Ramzi in “Preclinical Safety Studies of Glucose Oxidase,” J. Pharmacol. Exp. Ther. 266(3):1643-1648 (1993). Also, as reported by P. Heiss, S. Bematz, G. Bruchelt and R. Senekowitsch-Schmidtke in “Cytotoxic Effect of Immunoconjugate Composed of Glucose Oxidase Coupled to a Chimeric Anti-ganglioside (GD2) Antibody on Spheroids,” Anticancer Res. 15(6A):2438-2439 (1995). They report that the therapeutic use of the chimeric anti-ganglioside (GD2) antibody shows some success in the therapy of neuroblastomas and melanoma as shown in various Phase I studies. To enhance the effect, glucose oxidase is coupled to the anti-GD2 antibody to produce H₂O₂ in the presence of glucose and oxygen. H₂O₂ easily penetrates the target cells in contrast to the antibody.

Glucose oxidase may also be used for the production of antimicrobial products such as soaps and cremes; for example, Kitchen Cupboard Almond Milk Kitchenhand Creme 2 oz. contains glucose and glucose oxidase. Also, glucose oxidase may be used in synthetic saliva, such as Biotene and the like, since many saliva contain an optimum concentration of a natural enzyme system that regulates the microbiological oral ecosystem (glucose oxidase+lactoperoxidase system).

Biochemical applications could also include Immunochemistry. For example, Vector offers VECTASTAIN® ABC kits of peroxidase, alkalinephosphatase and glucose oxidase for immunohistochemistry, ELISAS and blot detection. It could also be used for identifying and/or tracking proteins as reported by J. J. Marchalonis in “Enzymatic lodination of Proteins,” Biochemical Journal, 113, 229-305, (1969) and by J. I. Thorell and B. G. Johansson, Biochemica et Biophysica Acta, 251,363-9, (1969).

Other uses could include enzymatically amplified sensors for amperometry and voltammetry including electrodes designed for amperometric detection of glucose. For example, enzyme reactions have been widely explored in combination with the electrode chemical techniques to add specificity to voltammetry and amperometry. Such strategies are often referred to as “biosensors” since they employ a biomolecule (e g. enzyme, antibody) and can be used for sensing purposes. The most common situation is to use an oxidase enzyme to detect its primary substructrate (e.g. glucose oxidase to detect glucose). The enzyme typically oxidizes the substrate and then transfers reducing equivalence (electrons) to a small molecule (acceptor or mediator) which can be oxidized at the electrode surface. Electrodes designed for the amperometric detection of glucose, lactate and cholesterol are common examples which have used this technique.

Research has been conducted to design various different types of enzyme electrodes. Using the analyte molecule functioning as a mediator, a saturating excess of the enzyme's substrate is used to make the reduced enzyme kinetically inexhaustible. Once an analyte molecule is oxidized at an electrode surface, it is rapidly reduced by the enzyme and is hence available for re-oxidation. This means that each analyte molecule is detected several times on the experimental time scale, thus, the analytical signal is chemically amplified by the enzyme reaction. For example, catechol analytes using glucose oxidase have been proposed.

Thus, a need exists to utilize the interactions of insect enzymes with plants to improve agriculture. Specifically, there is a need to develop methods of increasing systemic plant resistance to pathogens using recombinant DNA technology. It is desirable to develop recombinant vectors encoding an insect glucose oxidase that may be introduced into a variety of plant species.

There is also a continuing demand for alternative sources of glucose oxidase for various fields including biomedical, biochemical, food production and preservation, and the like. It is therefore desirable to develop methods of manufacturing large quantities of stable glucose oxidase.

### SUMMARY OF THE INVENTION

The present invention provides an isolated and purified DNA molecule comprising a DNA segment encoding a Helicoverpa zea, commonly known as the corn earworm, glucose oxidase (Gox) gene and methods for conferring enhanced resistance to insect predation by introducing and expressing this glucose oxidase gene in plant cells. The DNA molecule encoding Gox can encode an unaltered Gox or an altered Gox that substantially catalyzes the oxidation of glucose to form hydrogen peroxide. A DNA molecule of the invention may further comprise various known polynucleotide control sequences known to regulate the translation, transcription and function ofthe Gox gene. The polynucleotide sequence may also be encoded on an RNA molecule instead of DNA, as the nuleotide sequence is the significant aspect of the invention.

The present invention also discloses the amino acid sequence of the H. zea Gox protein that corresponds to the DNA sequence of the Gox Gene. As will be appreciated by those skilled in the art of biotechnology, this amino acid sequence may be altered by site-directed mutagenesis or enhanced by adding known amino acid sequences to either the N terminus or C terminus of the protein. For example, it is known that certain amino acid transit sequences direct a protein to specific areas within an organism. It is also well known in the art to add a polyhistidine sequence to a protein to facilitate purification. The addition of amino acid sequences to a known protein is usually accomplished by adding a DNA sequence upstream or downstream of the polynucleotide sequence that codes for the protein.

Insect salivary proteins may perform multiple physiological roles including induction or suppression of plant defense responses. The H. zea Gox was previously purified from salivary glands of the corn earworm and demonstrated to play an important role in inducing host resistance. The amino acid sequence of H. zea Gox is unique, sharing less than 30% homology with other reported Gox proteins. In particular, the novel protein structure shares very little homology with other proteins known to have the similar function of glucose oxidation. Because this protein has such a novel structure, it is unlikely that other glucose oxidases will confer the same type of resistance in plants. The corresponding Gox cDNA and two related genes from H. zea have been isolated. The Gox gene encodes a protein of 606 amino acids with an estimated molecular mass of 67 kD and a calculated isoelectric point of 5.26. The Gox gene is specifically expressed in the salivary gland of H. zea in a developmentally dependent manner. To determine the role of Gox in activation of host resistance, the Gox gene was introduced into tobacco plants via Agrobacterium-mediated transformation. Transgenic tobacco carrying the Gox transgene produces a large amount of Gox protein and exhibits high levels of Gox enzyme activity. Furthermore, the expression of Gox in transgenic tobacco was shown to significantly enhance host resistance against insect herbivory.

Expression of the H. zea Gox discourages predation of a transgenic plant by a variety of predators, including H. zea itself as well as other insects and pathogens. In addition, expression of Gox, even in relatively high sublethal concentrations, has no deleterious effects on the plant itself Plants grow at a normal rate into healthy plants. This makes the Gox gene especially well suited for creating transgenic commercial crops having increased resistance. Use of a recombinant Gox gene makes production of many crops, such as wheat, corn, soy beans, tomatoes and rice, both easier and cheaper.

The polynucleotide sequence encoding the Gox gene may also be used in microbial organisms, including bacteria and yeasts, to rapidly produce large quantities of Gox for other uses. Those skilled in the art of biotechnology will appreciate that various bacteria, such as E. coli, may be used as microbial factories to rapidly produce large amounts of a desired protein. It would be obvious to those skilled in the art that there are many ways to insert the disclosed Gox polynucleotide sequence into a bacteria and produce Gox on a large scale. H. zea Gox is a suitable protein for use in electrochemical sensing and analysis, measuring the level of sugar in blood or urine, voltammetry and any other application of a glucose oxidase.

Whether the Gox sequence is to be inserted into a transgenic plant to bolster its defenses to insect predation or placed in a microbe for large scale protein production, various known genetic sequences may be ligated both upstream and downstream from the Gox gene in order to regulate transcription and translation of the polynucleotide sequence. What additional nucleotide sequences are attached to the Gox sequence will depend on a wide variety of factors, such as the recombinant vector used, the type of cells into which the Gox gene is inserted, the method of inserting the genetic sequence, the desired level of expression of the Gox gene and other factors well known to those skilled in the art.

The present invention also discloses a recombinant antibody developed to ease detection of the protein. A significant portion of the Gox peptide sequence was used to develop an antibody that was shown to bind to the complete peptide and portions thereof Using the well known technique of an immunoblot assay, the presence of the protein may be readily detected.

It is therefore an object of the present invention to provide a polynucleotide sequence encoding an insect Gox protein that may be inserted into recombinant vectors that may be used to create transgenic plants having increased resistance to insect predation.

It is another object of the present invention to provide a polynucleotide sequence encoding an insect Gox protein that is suitable for large scale production in microbes of a glucose oxidase enzyme suitable for chemical, medical and industrial uses.

It is another object of the present invention to provide a polynucleotide sequence encoding an insect Gox protein that may be used in conjunction with know nucleotide sequences to enhance or modify translation and transcription of the Gox polynucleotide sequence.

It is another object of the present invention to provide recombinant vectors that may be used to insert a polynucleotide sequence coding for the Gox protein into a variety of organisms, including plants and microbes.

It is another object of the invention to provide antibodies that may be used to detect the presence of the Gox protein or a catalytically active portion thereof.

It is another object of the present invention to provide transgenic plants that express the Gox protein or a catalytically active portion thereof and exhibit enhanced resistance to insect predation.

### BRIEF DESCRIPTION OF THE DRAWINGS

FIG. 1 is a DNA blot illustrating the copy number of the Gox gene in H. zea genome. Total DNA was isolated from H. zea larvae by phenol/chloroform extraction. Ten microgram of genomic DNA was digested with EcoRI and HindIII, respectively, fractionated on a 1% agarose gel, and blotted onto a nylon membrane. The DNA blot was hybridized with a gene-specific probe (850˜900 bp of Gox) labeled with a PCR DIG Probe Synthesis Kit (Roche Molecular Biochemicals).

FIG. 2 are RNA blots illustrating the specific expression of the Gox gene in salivary glands during particular developmental stages. Total RNAs were isolated from the 6th instar H. zea at the different days using TRIzol reagent (Life Technology, Md.). Ten microgram of total RNA was separated on a 1.2% agarose gel containing formaldehyde and then transferred onto a nylon membrane. The RNA blots were hybridized with a gene-specific probe labeled with a PCR DIG Probe Synthesis Kit (Roche Molecular Biochemicals). The sample loading was verified by hybridizing with a labeled rDNA fragment from H. zea. A. RNA samples from salivary glands; B. RNA samples from whole larvae; C. RNA samples from the larvae from which salivary glands were removed.

FIG. 3 is an immunoblot illustrating the expression of the Gox protein in H. zea larvae. Total proteins were extracted from the 6th instar larvae. Twenty microgram of total protein extract was loaded onto each lane of 10% SDS-PAGE gel. After electrophoresis, separated proteins were transferred onto hybond-P PVDF membrane. The Gox protein was detected with the anti-Gox antibody using the ECL Plus detection system (Amersham).

FIG. 4 A. shows the transgenic tobacco carrying the 35S:Gox transgene. A. Morphologically normal transgenic plants; B. shows an immunoblot showing the overexpression of the Gox protein in transgenic plants as detected by immunoblot analysis with the anti-Gox antibody.

FIG. 5 is a graph showing the inhibitory effect of Gox expression in transgenic tobacco on larval growth of H. zea. Control: vector-transformed transgenics; Lox-Gox: transgenics with Gox levels less than 50 μmol/min/mg protein; high-Gox: transgenics with Gox levels higher than 100 μmol/min/mg protein.

FIG. 6 is a schematic diagram of a construct comprising the Gox gene and the PGEM-T Easy vector.

FIG. 7 is a schematic diagram of the pBK-CMV phagemid vector.

FIG. 8 is a schematic diagram of a construct comprising the BamH1/Sal1 portion of the Gox gene and the pET28a(+) vector.

FIG. 9 is a schematic diagram of a construct comprising Gox cDNA, the CaMV 35S promoter and the pCAMBIA vector.

### DETAILED DESCRIPTION OF THE PREFERRED EMBODIMENTS

The present invention provides a cDNA sequence encoding a glucose oxidase (Gox) from the corn earworm Helicoverpa zea. Knowledge of this sequence allows the expression in recombinant systems of polypeptides substantially similar to Gox, including Gox, analogs of Gox, and fragments of Gox.

The present invention also provides the amino acid sequences for which the Gox gene codes. Knowledge of these sequences allows the synthetic formation of these proteins, analogs of these proteins that perform the same catalytic function, and fragments of these proteins that induce the same enhanced insect resistance as the entire polypeptide.

In describing the present invention, the following terminology will be used in accordance with the definitions set out below. This terminology is well known to those skilled in the art.

“Glucose oxidase” or “Gox” refers to a polypeptide which catalyzes the oxidation of glucose to gluconic acid with the concomitant production of hydrogen peroxide. Procedures for determining glucose oxidase activity are known in the art.

“Recombinant polynucleotide” refers to a polynucleotide of genomic, cDNA, semisynthetic, or synthetic origin which, by virtue of its origin or manipulation: (1) is not associated with all or a portion of the polynucleotide with which it is associated in nature, and/or (2) is linked to a polynucleotide other than that to which it is linked in nature, or (3) does not occur in nature.

“Polynucleotide” refers to a polymeric form of nucleotides of any length, either ribonucleotides or deoxyribonucleotides. This term refers only to the primary structure of the molecule. Thus, the term includes double- and single-stranded DNA, as well as double- and single-stranded RNA. It also includes modified, for example by methylation, phosphorylation, and/or by capping, and unmodified forms of the polynucleotide.

“Replicon” refers to any genetic element, e.g., a plasmid, a chromosome, a virus, that behaves as an autonomous unit of polynucleotide replication within a cell; i.e., capable of replication under its own control.

“Vector” is a replicon in which another polynucleotide segment is attached, so as to bring about the replication and/or expression of the attached segment. Vectors may have one or more polynucleotide or recombinant polynucleotide and one or more control sequences.

“Control sequence” refers to polynucleotide sequences which are necessary to effect the expression and/or secretion of coding sequences to which they are ligated. The nature of such control sequences differs depending upon the host organism; in prokaryotes, such control sequences generally include promoter, ribosomal binding site, and terminators; in eukaryotes, generally such control sequences include promoters, terminators and, in some instances enhancers. In addition, in both prokaryotes and eukaryotes, some control sequences direct the expressed polypeptide to a particular location within the cell or region within a multicellular organism. The term “control sequences” is intended to include, at a minimum, all components whose presence is necessary for expression, and may also include additional polynucleotide sequences that influence the expression of a protein.

“Promoter” refers to a polynucleotide sequence upstream from an expressed polynucleotide. A promoter sequence signals the cellular machinery to express the polynucleotide downstream from it. Some promoters operate like a switch and only signal a cell to express a downstream polynucleotide under certain conditions, such as when the organism is under insect/pathogen attack, above a certain temperature, or in the presence of a particular chemical such as IPTG.

“Gene” is a polynucleotide sequence which is transcribed into mRNA and/or translated into a polypeptide when placed under the control of appropriate regulatory sequences. The boundaries of the gene are determined by a translation start codon at the 5′-terminus and a translation stop codon at the 3′-terminus. A gene can include, but is not limited to, mRNA, cDNA, and recombinant polynucleotide sequences.

“Host cells”, “microbial cells”, “cells” and other terms denoting microorganisms or higher eukaryotic cell lines cultured as unicellular entities, are used interchangeably, and refer to cells which can be, or have been, used as recipients for recombinant vector or other transfer polynucleotides, and include the progeny of the original cell which has been transfected. It is understood that the progeny of a single parental cell may not necessarily be completely identical in morphology or in genomic or total DNA complement as the original parent, due to accidental or deliberate mutation. Progeny of the parental cell which are sufficiently similar to the parent can be characterized by the relevant property, such as the presence of a nucleotide sequence encoding a desired peptide, are included in the progeny intended by this definition, and are covered by the above terms.

“Transformation” refers to the insertion of an exogenous polynucleotide into a microbial cell, or cells of a multcellular organism such as a plant, irrespective of the method used for insertion, for example, direct uptake, transduction, f-mating, particle bombardment or bacteria-mediated gene transfer. The exogenous polynucleotide may be maintained as a non-integrated vector, for example, a plasmid, or alternatively, may be integrated into the host genome.

“Polypeptide” refers to the amino acid product of a sequence encoded within a polynucleotide, and does not refer to a specific length of the product, thus, peptides, oligopeptides, and proteins are included within the definition of polypeptide. This term also does not refer to post-expression modifications of the polypeptide, for example, glycosylations, acetylations, phosphorylations, sialylations, and the like.

IA. Molecular Cloning of Gox and Related Genes from H. zea

Helicoverpa zea eggs and neonates were obtained from the insect rearing facility at the Department of Entomology, University of Arkansas. Salivary glands were removed by dissection from the 6th instar of H. zea and stored at −80 C. The Poly(A) mRNAs were prepared from salivary glands of 6th instar H. zea larvae and used as templates for polymerase chain reaction (PCR) amplification. To isolate the Gox cDNA, two nested oligonucleotide primers (forward, 5′-ATGATYYTBGCYCARCARGA; 5′-CARACYG TBGTBGARG GHGC) were designed based on the N-terminal sequence (MILAQQDX GXQTVVEGASILNSXTAX VXTY) of Gox. The N-terminal sequence was obtained previously from purified Gox protein isolated from H. zea salivary glands. A reverse oligonucleotide primer (5′-CADCCDCCVARRMCYTTDCC) was designed according to a relatively conserved region (GKVLGGS) of Gox proteins. The amplified Gox fragments (about 340 bp) were purified from a 2% agarose gel and cloned into pGEM-T Easy plasmid vector (Promega).

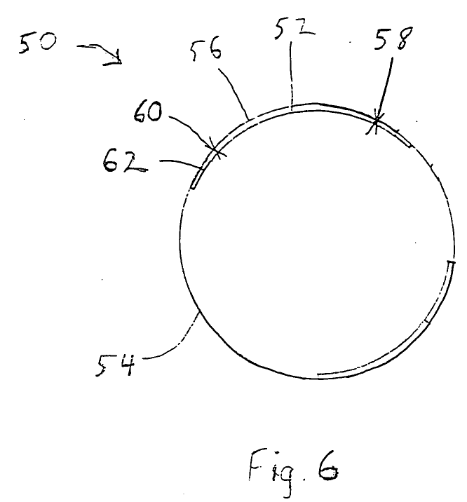

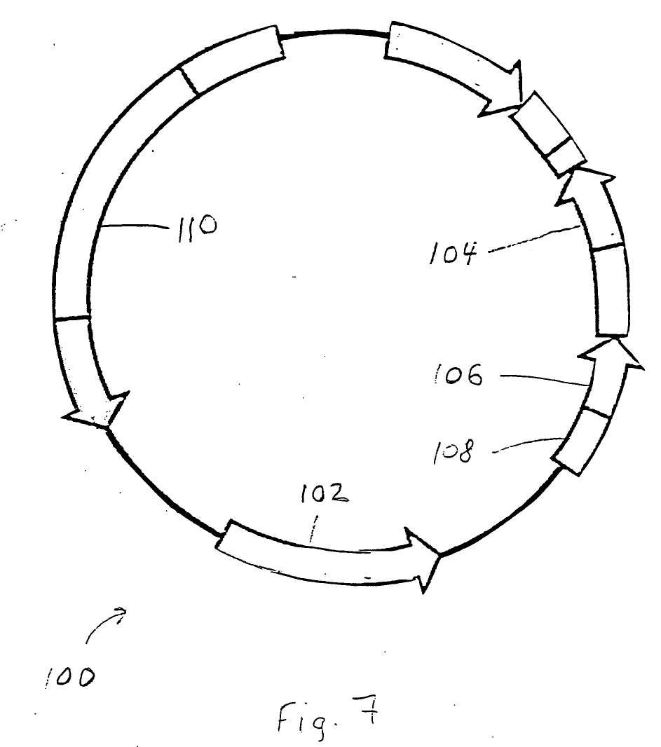

FIG. 6 illustrates construct 50 which comprises Gox fragment 52 and pGEM-T Easy plasmid vector 54. The vector 54 is spliced open at restriction sites 60 and 58, using restriction enzymes. The Gox fragment 52 was then inserted into the open frame 56 in vector 54 using a ligase enzyme. Promoter 62 is located immediately upstream of fragment 52 and regulates expression of fragment 52. Sequence analysis indicated that the 340 bp cDNA fragment encodes a 114 amino acid peptide that matches the N-terminal sequence and conserved domains of Gox.

Although pGEM-T Easy vector was used in this particular embodiment, those skilled in the art will appreciate that there are several vectors that may be used when isolating a polynucleotide, and the techniques employed in creating constructs such as that shown in FIG. 6 are common. Vectors are available commercially and many researchers develop their own vectors to suit their preferences. Which vector is used will depend on the desired method of transformation, the organism to be transformed, size of the polynucleotide, personal preference, and other factors known to those skilled in the art.

To further isolate full-length Gox cDNAs, an H. zea cDNA library was constructed with mRNAs from salivary glands. The salivary gland-specific cDNA library has an average insert size of about 1.5 kb and a phage titer of 5×109 pfu/ml. ZAP Express phage vector (Stratagene, Calif.) was used to construct the library. Again, those skilled in the art will appreciate that the ZAP Express vector is only one of several vectors suitable for constructing a cDNA library.

The 340 bp segment isolated during the initial PCR amplification was purified from the pGEM-T Easy vector and radiolabeled with ³²P by random priming. Radiolabeling polynucleotides is an ubiquitous process used in molecular biology. Those skilled in the art will appreciate that this is only one of several suitable methods of radiolabeling and that ³²P is only one of several suitable isotopes.

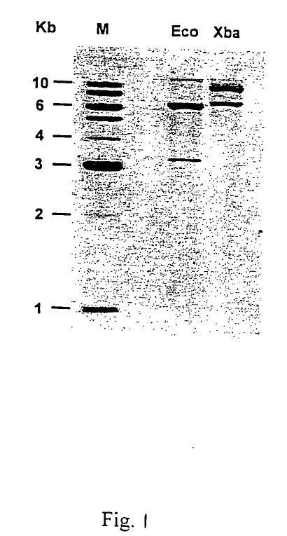

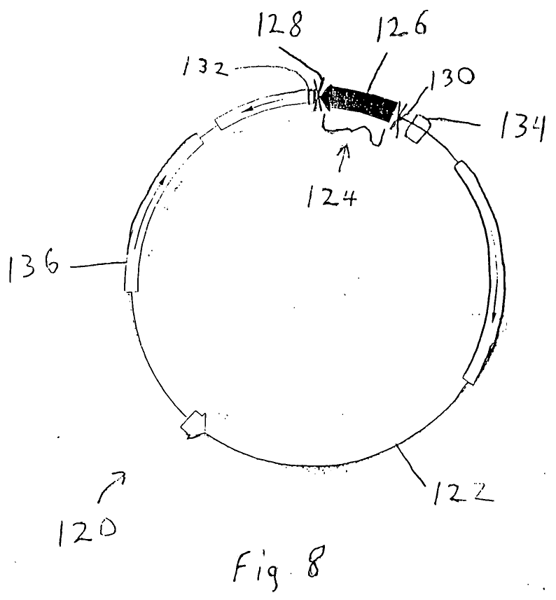

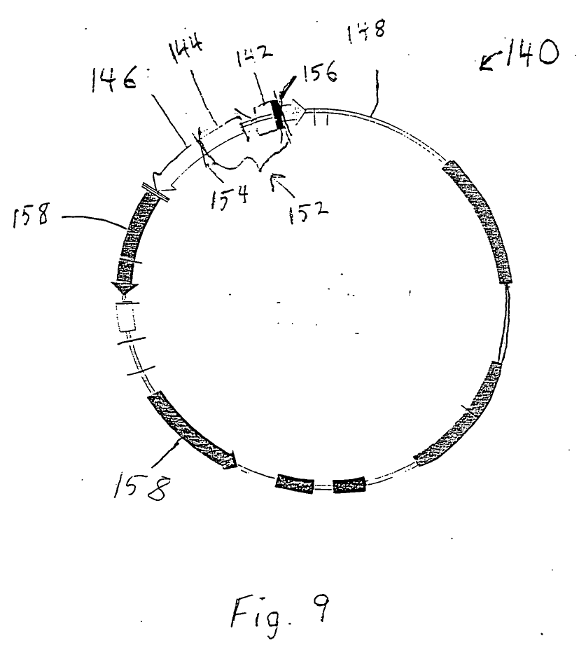

Using the radiolabeled 340 bp Gox fragment as a probe, a full-length cDNA of the Gox gene was isolated by library screening and subsequent excision of phagemid pBK-CMV carrying ihe Gox cDNA from ZAP Express. FIG. 7 shows pBK-CMV plasmid 100 having the ColE1 origin 102, the lacZ control sequence 104, lac' control sequence 106, CMV control sequence 108 and Neomycin resistance control sequence 110. The Gox cDNA fragment is 1958 bp long (SEQ ID NO: 1)and encodes a protein of 606 amino acid residues (SEQ ID NO: 4). The Gox protein has an estimated molecular mass of 67 kD, with a calculated isoelectric point of 5.26. The N-terminal sequence encoded by this gene is identical with that of purified H. zea Gox enzyme, confirming that it indeed codes for the same Gox enzyme identified by the biochemical analysis. Southern blot analysis reveals that there are two copies of the Gox gene in the H. zea genome (FIG. 1).

In addition to the Gox genes, at least two additional H. zea genes, HZCE15 (SEQ ID. NO: 2) and HzCE20 (SEQ ID NO: 3) that share significant sequence homology with Gox have been identified. The HZCE15 cDNA (1842 bp) encodes a protein of 583 amino acids (SEQ ID NO: 5) with an estimated molecular mass of 64 kD and a calculated isoelectric point of 5.37. The HzCE20 cDNA (839 bp) encodes a protein of 214 amino acids (SEQ ID NO: 6) with an estimated molecular mass of 24 kD and a calculated isoelectric point of 5.35. These Gox proteins show very little homology, less than 30%, with all known glucose oxidases from other species.

IB. Salivary Gland-Specific Expression of Gox in H. zea.

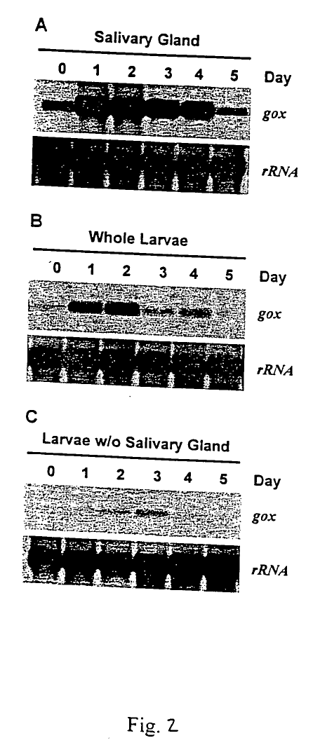

To examine the tissue specificity and developmental variation of Gox expression, RNA blot analysis was carried out using the 6th instar H. zea. As shown in FIG. 2A, the Gox gene is specifically expressed at high levels in salivary glands. The Gox transcripts are also present in whole larvae (FIG. 2B), but they are hardly detectable in the larvae from which the salivary glands were removed (FIG. 2C). The expression of Gox is low at day 0 and 5 of the 6th instar, peaks at day 1 and 2, and decreases at day 3 and 4 (FIG. 2A and 2B).

The fact that Gox is found almost exclusively in H. zea salivary glands is a strong indication that the plant defense mechanisms induced by Gox evolved as a response to predation by the corn earworm.

IC. Anti-Gox Antibody and Immunoblot Analysis.

To obtain recombinant Gox antigen for antibody production, the BamHI/SalI fragment (from 232 to 917 bp) of the Gox cDNA was fused in frame to the BamHI/SalI site of pET28a(+) vector (Novagen). FIG. 8 illustrates construct 120 that comprises plasmid vector pET28a(+) 122 containing the BamHI1/SalI fragment 126. The vector 122 is spliced open at restriction sites 128 and 130, using restriction enzymes BarrHi and Sall respectively. The Gox fragment 126 was then inserted into the open frame 124 in vector 122 using a ligase enzyme. JPTG-inducible promoter 134 is located immediately upstream of the BamHI/SalI fragment 126 and regulates its expression. Control sequence 132 adds a polyhistidine sequence to fragment 126 to facilitate purification. Control sequence 136 confers kanamycin resistance to the plasmid 122. By adding kanamycin to the media in which the host E. coli cells are grown, only bacteria that were successfully transformed will survive. Those skilled in the art will appreciate that this technique of plasmid construction is well known and that a wide variety of vectors are just as suitable as pET28a(+).

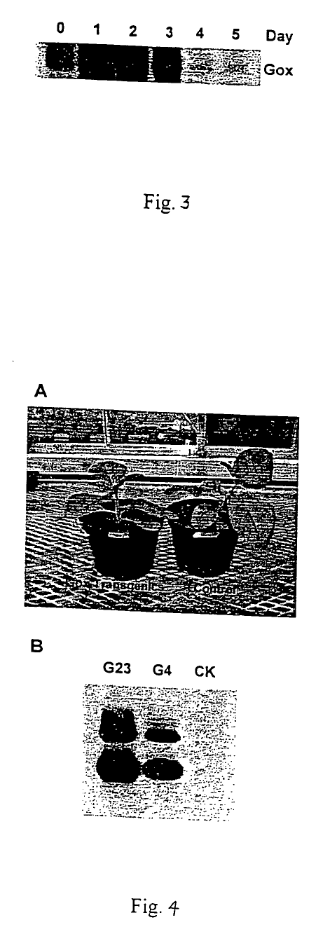

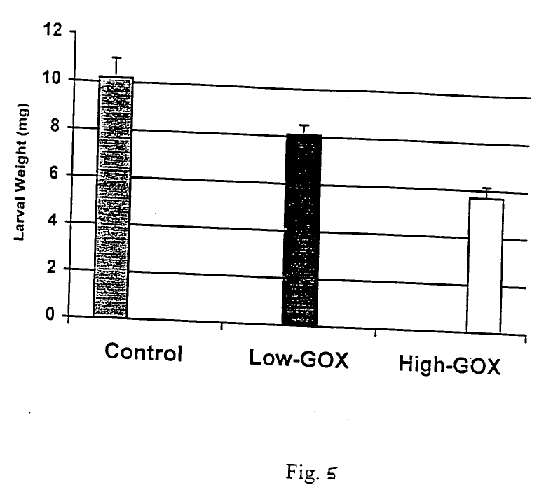

Cells of E. coli BL21 ([)E3) strain transformed with the recombinant plasmid was grown at 37 C in Luria-Bertani medium containing 50 ug/ml kanamycin in order to select for only bacterial cells that were transformed. The polyhistidine-tagged Gox peptide was induced in mid-logarithmic bacterial cultures by addition of 1 mM IPTG. After 3 h growth, bacterial cells were harvested and the recombinant Gox protein was purified using B-PER 6× His Spin Purification Kit (Pierce). The purified protein was subsequently used for the production of rat anti-Gox antibody. Immunoblot analysis reveals that the anti-Gox antibody specifically detects the Gox protein in H. zea. During the stage of the 6th instar, the Gox protein level is very high at day 1, 2 and 3, and drastically decreases at day 4 (FIG. 3). These data are consistent with high Gox enzyme activities previously found in salivary glands during 1st, 2nd and 3rd days of the 6th instar (Eichenseer et al., 1999).

Glucose Oxidase Expression in Tobacco Transgenics

HIA. Generation of Transgenic Tobacco Expressing Glucose Oxidase

To produce transgenic tobacco with overexpression of Gox, the full-length Gox cDNA was placed under the control of a double CaMV 35S promoter and cloned into binary vector pCAMBIA 2300 (CAMBIA, Australia). FIG. 9 illustrates construct 140 which comprises Gox cDNA 142, inserted CaMV 35S promoter 144 and pCAMBIA2300 plasmid 148. Plasmid 148 was spliced open at restriction sites 154 and 156 using restriction enzymes. Promoter 144 and Gox cDNA 142 were then spliced together and inserted into open frame 152. The pCAMBIA plasmid contains an additional CaMV35S promoter 146 upstream of a Kanamycin resistance control sequence 158.

Those skilled in the art will appreciate that which vector and promoter are chosen for a particular construct depend on a variety of factors, including but not limited to the desired level of expression of the inserted gene, the plant into which the vector is to be inserted, and the method employed to insert the gene and create a transgenic plant. Other vectors suitable for introduction of the Gox gene into other organisms include but are not limited to pBI101 series, pBIN19, pGA482, and pRT-100 series.

The resulting construct and empty vector control were individually introduced into Agrobacterium tumafaciens strain EHA105. Twenty-six primary (T0) transgenic plants were generated via the Agrobacterium-mediated transformation. These transgenic plants exhibit normal growth and produce high levels of Gox protein (FIG. 6). The Gox transgene appears to encode two peptides with different molecular weights, which results from alternative translations. In addition to the expression of Gox protein, many transgenic plants show very high levels of Gox enzyme activity (Table 1). The Gox activity can be inherited from T0 plants to their progeny (T1 plants) as shown by five representative transgenic lines.

In this particular embodiment, Agrobacterium-mediated transformation was used to create transgenic plants. However, those skilled in the art will appreciate that a variety of transformation techniques are suitable for introducing a Gox polynucleotide sequence into a plant. These alternatives include, but are not limited to electroporation, microinjection, protoplast transformation, liposomal encapsulation, and microprojectile bombardment.

TABLE 1
Glucose oxidase activity in selected transgenic lines (T0)
and their progeny (T1).

| Transgenic Lines | Gox activity (μmol/min/mg protein) |
|---|---|
| G4-T0 | 189 |
| G4-T1 (three progeny) | 160, 87, 103 |
| G11-T0 | 219 |
| G11-T1 (three progeny) | 50, 20, 35 |
| G16-T0 | 129 |
| G16-T1 (three progeny) | 38, 19, 17 |
| G24-T0 | 47 |
| G24-T1 (three progeny) | 15, 33, 22 |
| G26-T0 | 63 |
| G26-T1 (three progeny) | 22, 19, 27 |

IIB. Transgenic Tobacco Exhibits Enhanced Insect Resistance.

To determine the effect of Gox expression in transgenic tobacco on insect resistance, feeding assays were conducted using two-day-old first instar H. zea larvae. Terminal leaves of transgenic tobacco plants were excised from 6-8 node stage plants grown in a greenhouse. Leaves were placed with moistened filter paper in 16 oz plastic deli tub containers. Six transgenic lines each were used for the vector-transformed control, low Gox (<50 μmol/min/mg protein) transgenics, and high Gox (>100 μmol/min/mg protein) transgenics. For each transgenic line, 20 larvae were placed in each container. After 4 days larvae were individually weighed to the nearest 0.1 mg.

As shown in FIG. 5, larvae grown on Gox transgenic plants showed significantly reduced weight gain compared to the wild type controls. Larval growth was more than 40% suppressed in the transgenic lines with the highest Gox activity (>100 μmol/min/mg protein). Our data demonstrate that expression of Gox in transgenic tobacco significantly enhance insect resistance.

In the present embodiment, a transgenic tobacco plant was created. However, those skilled in the art will appreciate that this same technique may be used on practically any other plant and will result in substantially the same enhanced insect resistance. Such other plants include, but are not limited to, wheat, rice, corn, potatoes, tomatoes, peas, soy beans, lettuce, melons, green beans, squash, and broccoli. Those skilled in the art will understand that this process would be suitable and advantageous for any commercially grown crop.

Promoters, a subclass of control sequences, are required in order for a polynucleotide to be expressed. There are many known promoters. Which promoter is best for a given transgenic organism will depend on the desired level of expression and the type of organism being transformed.

Those skilled in the art will appreciate that in addition to the wide variety of vectors available for the techniques described herein, there are also a wide variety of control sequences that may be added to a polynucleotide sequence for a variety of reasons. It is possible that in some or all plants the defensive response induced by Gox will be enhanced by directing the Gox protein to a specific location within the plant. This may be accomplished using control sequences that result in the addition of amino acids at either the N-terminus or C-terminus of the Gox protein. These added amino acids utilize mechanisms within a plant to direct the protein to which they are attached to specific regions of the plant cell. For example, some control sequences direct proteins to the chloroplasts. Some control sequences result in the protein attaching to a membrane. The techniques of utilizing theses control sequences to direct a certain protein to a certain location are well known to those skilled in the art.

It is also well known to those skilled in the art that control sequences may also be used to regulate both the translation and transcription of a polynucleotide sequence. These control sequences may be employed to regulate the concentration of the protein within the organism that is expressing it. The addition of these various types of control sequences to any given vector is a relatively simple procedure.

Some control sequences require the addition of a second, regulatory gene. For example, some control sequences inhibit gene translation only when an inhibitor protein is present. In this situation, it is necessary to add the gene that encodes the inhibitor protein to the vector. This inhibitor protein gene may in turn have its own control sequences upstream or downstream from it. It is even possible for an inhibitor protein gene to have a control sequence that requires a second inhibitor protein gene in order to function properly. However, this is generally not desirable because the more complex a system is, the more likely it is to fail. In addition, just as there are control sequences that, require inhibitor proteins, there are also control sequences that require activation proteins that increase gene translation. These control sequences require the addition of an activation protein gene.

There are also control sequences that regulate expression of coding sequences at the transcription stage. These sequences inhibit or facilitate ribosomal activity on mRNA. All of these, mechanisms are well known to those skilled in the art.

Which control sequences, promoters and plasmids are used for a particular plant will be depend on the method of transformation, the plant into which the vector is introduced and personal discretion. Another significant factor is the fact that hydrogen peroxide is harmful and even lethal to plants when present in large amounts. Greater expression of Gox leads to greater insect resistance. However, too high a level of Gox expression within a plant could lead to harmful amounts of hydrogen peroxide. Appropriate promoters and control sequences used in conjunction with a Gox polynucleotide sequence will induce expression of enough Gox to enhance insect resistance, but limit production of the Gox protein so that harmful levels of hydrogen peroxide are not produced. A secondary factor to consider when determining the appropriate amount of Gox expression will be the amount of glucose present in the environment into which the plant will be placed. This is due to the fact that increased concentrations of a substrate increases catalytic activity of an enzyme.

One method of insuring that Gox is expressed in acceptable amounts is to use a promoter that is induced by insect predation. Such a promoter assures that the Gox gene is only expressed when the plant has been attacked by feeding insects.

It may also be desirable to use only a portion of Gox gene or protein. Sometimes a protein is more effective for a particular purpose when only a fragment is used. This can be accomplished by inserting only a portion of the encoding polynucleotide sequence, or by using only a fragment of the protein itself Only a portion of the Gox protein is necessary to induce enhanced insect resistance. Similarly, only a portion of the Gox protein is necessary to catalyze the oxidation of glucose. In some situations it may be advantageous to use only the necessary portion of the polynucleotide or polypeptide rather than the entire sequence. This use of a protein fragment is well known to those skilled in the art.

Whereas, the present invention has been described in relation to the drawings attached hereto, it should be understood that other and further modifications, apart from those shown or suggested herein, may be made within the spirit and scope of this invention.

What is claimed is:

1. A polynucleotide having the sequence of SEQ ID NO: 1 and conservatively modified variants thereof.

2. The polynucleotide of claim 1 wherein said polynucleotide is DNA.

3. A polynucleotide having the sequence of SEQ ID NO: 2 and conservatively modified variants thereof.

4. The polynucleotide of claim 3 wherein said polynucleotide is DNA.

5. A polynucleotide having the sequence of SEQ ID NO: 3 and conservatively modified variants thereof.

6. The polynucleotide of claim 5 wherein said polynucleotide is DNA.

7. A glucose oxidase protein having the amino acid sequence of SEQ ID NO: 4 and conservatively modified variants thereof.

8. A glucose oxidase protein having the amino acid sequence of SEQ ID NO: 5 and conservatively modified variants thereof.

9. A glucose oxidase protein having the amino acid sequence of SEQ ID NO: 6 and conservatively modified variants thereof.

10. A recombinant polynucleotide comprising a vector and a gene wherein said gene encodes an insect glucose oxidase protein.

11. The polynucleotide vector of claim 9 wherein said gene is selected from the group consisting of SEQ ID NO: 1, SEQ ID NO: 2, and SEQ ID NO: 3.

12. The recombinant polynucleotide of claim 9 wherein said vector is selected from the group cosisting of pGEM-T Easy, pBK-CMV, pET28a(+), pCAMBIA, pBI101, pGA482 and pRT-100.

13. A method for producing large quantities of the polypeptide of claim 5 comprising:
introducing the recombinant polynucleotide of claim 9 into host cells so as to yield transformed host cells;
inducing expression the protein encoded by said polynucleotide sequence; and, extracting said polypeptide from said host cells.

14. A method for creating a plant having enhanced resistance to insect predation comprising:
introducing the recombinant polynucleotide of claim 9 into the cells of a plant so as to yield transformed plant cells; and,
regenerating said transformed plant cells to provide a differentiated plant wherein said plant expresses the polypeptide encoded by said gene.

15. The transgenic plant formed by the method of claim 14.

16. The transgenic plant of claim 15 wherein said plant is a commercially grown crop.

17. The transgenic plant of claim 16 wherein said plant is tobacco.

18. A seed of the transgenic plant formed by the method of claim 14.

19. An antibody that binds specifically to an insect glucose oxidase protein.

20. The antibody of claim 19 wherein said glucose oxidase protein is selected from the group consisting of SEQ ID NO: 4, SEQ ID NO: 5, and SEQ ID NO: 6.


SEQUENCE LISTING (6 sequences)

SEQ ID NO: 1 - DNA, 1958 bp, Helicoverpa zea
  CDS 15..1833
taggaaaataccaagatgattctggcgcagcaagattgcggctgccaaacagtagtagaaggtgccagtatcctgaactccacagcatgtagcggcacctatctgttcatggtactcctacaagggtacttatggggccgctgtgaaatcgccacgccttgcaagagaatcgaatccatagatgaaacagagtcagaatatgacttcatagtagtgggagctgggtcttcaggatccattgtagctgggagactgagcgaaaatactacatataaagtccttctattagaagctggtgggcccgaacctctgggtgctcgtgtcccatcattttacaaaactttctggggccacgatgaggtagactggcaagggcgagccgtacctgatcccaacttctgccgcgaccaaggagaactcgggtgccaatggccgctaggaaaaagcttaggaggatctagtctcctcaacggtatgatgtaccacaaaggccacgccgctgactacgagacctgggtggaagaaggcgcggaaggttggtcctgggacgaggtcaaaccattcatggacttggccgaaggtaacagacaagtgggaagcctcgtcgaagggaagtaccactccgaaactggacgcatgccaatacaaacatttaactaccagcccccgcagcttagggatctaatagaagcaatcaaccagacgggactgccgatcatcacggacatgaacaacccgaacacacccgacggctttgtagtagcacaaacctttaatgacaatggccagcgctacaccacagcccgcgcatacctggctcccaaatccgagcgacccaacctgagcgtcaaactgtacgcccacgtgactaaagtactcttcgacgggaagaaggctgtgggagtggaatacgtcgacaagaacggcaatacgaaaactgtcaagactactaaagaggtaatcgtgtcagcaggaccattgaccagtcctaaaatcctgatgcattctggtgtgggacctaaagaggtattagagccattgggcattcccgtagtagctgacgtgcccgtcggtaagaggctgaggaaccactgcggggccacgctgaacttcctcctgaagaagtccaacaacacccagtctttggactggagtgcactgactgactacttgctggaacttgacgggcctatgagttccactgggcttactcagctcactggcctcctatactcaagctacgcagacaagagtcgcaagcagccagacctacagttcttcttcaacggtctgtatgctgactgctccaagactggtgttatcggcgaaccggctgaggactgcagcgatggctacaaaatctcagcgaatgccgtagccctgctcccgcgcagcgtgggccacgtgaccatcaactcgacagaccccttcaagtcagcgctgttctaccccaacttcttctctcacccagacgacatgaacatcgtgatggaaggcgttgattacttacgcaagattttcgaaagtcaggtgctacaagagaaatacaaagtagaactagacccggaatacacacaagagtgtgacgactacaaagcgtggtcaagagactggaaggagtgtatgattcgcctacacactgacccacagaaccatcagctcgccaccaacgctattggcaaggtcgttgacccgcagcttagggtttatgacgtcaaaaacttgcgagtatgtgacgcgggttcaatgccctcaccgccgaccggcaaccctcaaggcgctatcatggtggtagcagaacgatgcgcacacttcatcaaacagacttggcaatagaaccacgagcttacaacatataccctatgcaatgttatccttgttattatcaattacgtacataaagtacgagaattatgttcctcttccagtgaataaaatgttgaatatttaaaaaaaaa

SEQ ID NO: 2 - DNA, 1842 bp, Helicoverpa zea
  CDS 17..1765
gtcacatctcacagcaatggcagacgcggctgcttcccagtcatcggcgcagacggtcaagcttgctctgcaagtgctgcagacgctgagcctcaccgcgtggcagtacccgcctgactgcgctctcactaatgggagctcattcgacttcatagtggtggggagtggcaccgctgggtcagtgctggccaacaggctgtcagccaacgatagtgtcagtgtgttgctccttgaagcgggaggatatccaccattggagtcagagctgcctgcgcttttcatgatgttaagcaactcggactatgactacaaatactacgcagaaaacgacaactacacgatgcagaacatacgagggaagagatgtgccctcactcaaggcaaggtgttgggaggcaccagctccacctatgccatgatgcatacgagaggtgatcctcaggactacgacgtgtgggcggagagggccaacgatacaacctggaatgccaccaacacattgtcatatttcaagaaacaagaaaaactaactgacgaagaactcctgcactctgagtacgctgctgtccacggtactgatgggatggtcaaaataagaagagagacaagtcctcttctcgatgatattcttggacgcattttaagagggcgggcacgatttaatgatggacaccacatcattgaaagtcttcgcttcggctacacacaggttgctatatgccatcgaatgatggagtgcgggcaaagtagtgcacttgcctatctaagttctgccaagaaacgaaagaatctatgtgtatctttatttactactgctaccaagatcttgatcgagaatgaggtcgctgttggtgtacagctgacgacctctaccaacgaaacatacaatatatattccaacaaggaagtgattgtgtctgctggcaccttcaacagtcccaagctactgatgctgtctggcattggaccccgcgaacatttggagtcagtcgaaatcgacgtggttgctgaccttccagtcggccagaactacatggatcagccgagtgctccaattattatccaaatggatgaaagtgcagaagtagctggtgccataaacccacaccagttccctctgccaactttcattgggaacgtagccctcgattcacccagcaagcgaccacaataccacacggtcaattttctgttcccggcgaactccacagatctactggatatgtgttccttgtttcttagttactcggatgaagtttgccagaaggtctacgaggcgaccacgaaccgaaagactatattttccttggtagggttggcgttaccaaactctagaggtgaagtattgctggccagcgctgaccctgctgcagcaccaatcgttcacactggcatgttcagcaactatacggacttgaacctgatggggcgcgcttttatcgaccacgtaagagtgctgaactctacttacttccgaagcgtaaacgcgacaatcttggaccttggcttctgcaaggatacaacaagtgaggtggaattctgggagtgctacacgttggctatgtcgaataccatgtggcactttggagggacttgtgccatgggtctggtgctggacagcaagatgaaggtcaaaggcgtggggaggctgagggtggtggactcctcctccatgccggcgcttgttaccgggaaggtcaactctccgatcggcatgcttgctgaaaaagctgctgatttcatactaactgatcataacatcttgtaactctcttgaaacaattacaatttggtcaataaaaattaaatgagttaaaacgtgaaaaaaaaaaaaaaaaaaaa

SEQ ID NO: 3 - DNA, 824 bp, Helicoverpa zea
  CDS 63..704
acaacacccagtctttggactggagtgcactgactgactacttgctggaacttgacgggcctatgagttccactgggcttactcagctcactggcctcctatactcaagctacgcagacaagagtcgcaagcagccagacctacagttcttcttcaacggtctgtatgctgactgctccaagactggtgttatcggcgaaccggctgaggactgcagcgatggctacaaaatctcagcgaatgccgtagccctgctcccgcgcagcgtgggccacgtgaccatcaactcgacagaccccttcaagtcagcgctgttctaccccaacttcttctctcacccagacgacatgaacatcgtgatggaaggcgttgattacttacgcaagattttcgaaagtcaggtgctacaagagaaatacaaagtagaactagacccggaatacacacaagagtgtgacgactacaaagcgtggtcaagagactggaaggagtgtatgattcgcctacacactgacccacagaaccatcagctcgccaccaacgctattggcaaggtcgttgacccgcagcttagggtttatgacgtcaaaaacttgcgagtatgtgacgcgggttcaatgccctcaccgccgaccggcaaccctcaaggcgctatcatggtggtagcagaacgatgcgcacacttcatcaaacagacttggcaatagaaccacgagcttacaacatataccctatgcaatgttatccttgttattatcaattacgtacataaagtacgagaattatgttcctcttccagtgaataaaatgttgaatatttaaaa

SEQ ID NO: 4 - protein, 606 aa, Helicoverpa zea
MILAQQDCGCQTVVEGASILNSTACSGTYLFMVLLQGYLWGRCEIATPCKRIESIDETESEYDFIVVGAGSSGSIVAGRLSENTTYKVLLLEAGGPEPLGARVPSFYKTFWGHDEVDWQGRAVPDPNFCRDQGELGCQWPLGKSLGGSSLLNGMMYHKGHAADYETWVEEGAEGWSWDEVKPFMDLAEGNRQVGSLVEGKYHSETGRMPIQTFNYQPPQLRDLIEAINQTGLPIITDMNNPNTPDGFVVAQTFNDNGQRYTTARAYLAPKSERPNLSVKLYAHVTKVLFDGKKAVGVEYVDKNGNTKTVKTTKEVIVSAGPLTSPKILMHSGVGPKEVLEPLGIPVVADVPVGKRLRNHCGATLNFLLKKSNNTQSLDWSALTDYLLELDGPMSSTGLTQLTGLLYSSYADKSRKQPDLQFFFNGLYADCSKTGVIGEPAEDCSDGYKISANAVALLPRSVGHVTINSTDPFKSALFYPNFFSHPDDMNIVMEGVDYLRKIFESQVLQEKYKVELDPEYTQECDDYKAWSRDWKECMIRLHTDPQNHQLATNAIGKVVDPQLRVYDVKNLRVCDAGSMPSPPTGNPQGAIMVVAERCAHFIKQTWQ

SEQ ID NO: 5 - protein, 583 aa, Helicoverpa zea
MADAAASQSSAQTVKLALQVLQTLSLTAWQYPPDCALTNGSSFDFIVVGSGTAGSVLANRLSANDSVSVLLLEAGGYPPLESELPALFMMLSNSDYDYKYYAENDNYTMQNIRGKRCALTQGKVLGGTSSTYAMMHTRGDPQDYDVWAERANDTTWNATNTLSYFKKQEKLTDEELLHSEYAAVHGTDGMVKIRRETSPLLDDILGRILRGRARFNDGHHIIESLRFGYTQVAICHRMMECGQSSALAYLSSAKKRKNLCVSLFTTATKILIENEVAVGVQLTTSTNETYNIYSNKEVIVSAGTFNSPKLLMLSGIGPREHLESVEIDVVADLPVGQNYMDQPSAPIIIQMDESAEVAGAINPHQFPLPTFIGNVALDSPSKRPQYHTVNFLFPANSTDLLDMCSLFLSYSDEVCQKVYEATTNRKTIFSLVGLALPNSRGEVLLASADPAAAPIVHTGMFSNYTDLNLMGRAFIDHVRVLNSTYFRSVNATILDLGFCKDTTSEVEFWECYTLAMSNTMWHFGGTCAMGLVLDSKMKVKGVGRLRVVDSSSMPALVTGKVNSPIGMLAEKAADFILTDHNIL

SEQ ID NO: 6 - protein, 214 aa, Helicoverpa zea
MSSTGLTQLTGLLYSSYADKSRKQPDLQFFFNGLYADCSKTGVIGEPAEDCSDGYKISANAVALLPRSVGHVTINSTDPFKSALFYPNFFSHPDDMNIVMEGVDYLRKIFESQVLQEKYKVELDPEYTQECDDYKAWSRDWKECMIRLHTDPQNHQLATNAIGKVVDPQLRVYDVKNLRVCDAGSMPSPPTGNPQGAIMVVAERCAHFIKQTWQ